(19) United States
(12) Patent Application Publication
(10) Pub. No.: US 2007/0212773 A1
(43) Pub. Date: Sep. 13, 2007

(54) Electrical signal measuring device for cells in culture and electrical signal measuring method that uses same device

(76) Inventors: Teruo Fujii, Tokyo (JP); Yasuyuki Sakai, Tokyo (JP); Serge Ostrovidov, Tokyo (JP)

Correspondence Address: KENYON & KENYON LLP, ONE BROADWAY, NEW YORK, NY, 10004, US

(21) Appl. No.: 11/707,588

(22) Filed: Feb. 15, 2007

(30) Foreign Application Priority Data
Feb. 16, 2006 (JP) 2006-039230

(51) Int. Cl. C12M 1/34

(52) U.S. Cl. 435/287.1; 435/297.1; 435/297.2

(57) ABSTRACT

An electrical signal measuring device that can measure electrical characteristics of cells in culture in real time and with high accuracy while maintaining them in a favorable state and an electrical signal measuring method that uses the device are disclosed. The electrical signal measuring device 1 for cells in culture includes a support body 2, a compartment in the support body, a semipermeable membrane 3, and electrodes, in which the compartment is divided by the semipermeable membrane 3 into an upper compartment 4 and a lower compartment 5; an upper electrode 61 is provided in the upper compartment 4, and a lower electrode 62 that faces the upper electrode 61 and whose facing surface 621 is in contact with the semipermeable membrane 3 is provided in the lower compartment 5; and an upper perfusion channel and a lower perfusion channel are provided in the support body 2 to separately perfuse fluid in the upper compartment 4 and the lower compartment 5 is disclosed.

### BACKGROUND OF THE INVENTION

1. Field of the Invention

The present invention relates to an electrical signal measuring device that can measure electrical current responses in real time and with high accuracy such as impedance and the like when an electrical field is impressed on cells in culture, and to an electrical signal measuring method that uses the same device.

Priority is claimed on Japanese Patent Application No. 2006-039230, filed Feb. 16, 2006, the content of which is incorporated herein by reference.

2. Description of Related Art

Analyzing adhesive cells in culture or the state of tissue formed by said cells in culture noninvasively and in real time is an extremely effective means when performing analysis of the onset mechanism of various diseases or development of new drugs. In order to obtain more useful information, it is necessary to be able to perform analysis with high accuracy at a micro level. In fields related to medical science and drug development, the development of such a microfluidic device that allows such analysis is strongly desired.

Generally, in order to analyze cells in culture or the structure and internal state of tissue formed by said cells in culture in detail, it is required to both maintain the cells in culture in a favorable state during analysis and adopt a high-precision analytical method.

Methods that, for example, measure in real time the electrical resistance or the electrical impedance of cells in culture have conventionally been adopted as analytical methods. In particular, methods that measure the impedance of cells in culture are effective since a greater amount of information relating to the cells in culture is obtained noninvasively and in real time. As a method of measuring the impedance of such cells in culture, there is disclosed a method that uses a device with patterned electrodes on a thin film flat surface by microfabrication. (Refer, for example, to non-patent document 1).
Non-Patent Document 1: Linderholm, P., Brouard, M., Barrandon, Y., Renaud, P. Monitoring stem cell growth using a microelectrode array. XII ICEBI 2004 Gdansk.

However, conventional methods such as the method disclosed in non-patent document 1 cannot achieve both maintenance of cells in culture in a favorable condition and high-precision analysis, since their accuracy is insufficient to obtain more detailed information. This is because the device does not reflect environment conditions in vivo, thereby cannot maintain cells in culture in a favorable state since its environment differs greatly from in vivo conditions, and because the shape and arrangement of the electrodes used for the measurement of impedance and the like are not suited to measure faint electrical signals.

The present invention, achieved in view of the aforedescribed circumstances, has as its object to provide an electrical signal measuring device that can measure electrical characteristics of cells in culture in real time and with high accuracy while maintaining them in a favorable state and provide an electrical signal measuring method that uses the device.

The present inventors have arrived at the present invention as a result of concerted study, with the discovery that electrical characteristics of cells in culture can be measured in real time and with high accuracy while maintaining them in a favorable state by developing a cell-culturing device that can closely reproduce an in vivo environment, culturing cells on a semipermeable membrane provided in the device, arranging two electrodes so as to locate each of the electrodes above and below the cells in culture on the semipermeable membrane, and impressing an electrical field on the cells in culture.

### SUMMARY OF THE INVENTION

The first aspect of the present invention is an electrical signal measuring device for cells in culture including a support body, a compartment in the support body, a semipermeable membrane, and electrodes, in which the compartment is divided by the semipermeable membrane into an upper compartment and a lower compartment; an upper electrode is provided in the upper compartment, and a lower electrode that faces the upper electrode and whose facing surface is in contact with the semipermeable membrane is provided in the lower compartment; and an upper perfusion channel and a lower perfusion channel are provided in the support body to separately perfuse fluid in the upper compartment and the lower compartment.

Preferably the electrical signal measuring device for cells in culture is characterized by the distance between the surface of the upper electrode that faces the lower electrode and the surface of the semipermeable membrane that faces the upper electrode being 1 mm or less.

Preferably the electrical signal measuring device for cells in culture is characterized by the thickness of the measurment surface of the upper electrode and the lower electrode being 0.5 mm or less.

Preferably the electrical signal measuring device for cells in culture is characterized by the material of the upper electrode and the lower electrode being gold-plated brass, and the shape of the measuring surface of these electrodes being circular with a diameter of 3 mm or less.

Preferably the electrical signal measuring device for cells in culture is characterized by the material of the support body being polydimethylsiloxane.

Preferably the electrical signal measuring device for cells in culture is an impedance measuring device.

Preferably another aspect of the present invention is an electrical signal measuring array for cells in culture characterized by that the electrical signal measuring device for cells in culture being plurality provided on a base material.

Preferably another aspect of the present invention is a method of measuring electrical characteristics of cells in culture using the electrical signal measuring device for cells in culture, including: introducing cells into a location located between the upper electrode and the lower electrode on the upper compartment side of a semipermeable membrane; culturing the cells on the semipermeable membrane while separately perfusing a fluid in the upper compartment and a culture medium in the lower compartment; impressing an electrical field on the cells in culture by the upper electrode and the lower electrode; and measuring the current response of the cells in culture to the impressed electrical field.

The present invention can measure electrical characteristics of cells in culture in real time and with high accuracy while maintaining them in a favorable state.

### BRIEF DESCRIPTION OF THE DRAWINGS

FIG. 1 is a longitudinal sectional view that shows one example of the electrical signal measuring device for cells in culture of the present invention.

FIG. 2A is graph that shows the impedance measurement result of cells in culture in example 1 which shows the amplitude spectra of the measured impedance.

FIG. 2B is graph that shows the impedance measurement result of cells in culture in example 1 which shows the phase spectra of the measured impedance.

FIG. 3A is a graph that shows the impedance measurement result of cells in culture in example 2 which shows the amplitude spectra of the measured impedance.

FIG. 3B is a graph that shows the impedance measurement result of cells in culture in example 2 which shows the phase spectra of the measured impedance.

### DETAILED DESCRIPTION OF THE INVENTION

The present invention will be described hereinbelow with reference to the attached drawings. Note that the present invention is not in any way limited to the embodiments illustrated hereinbelow.

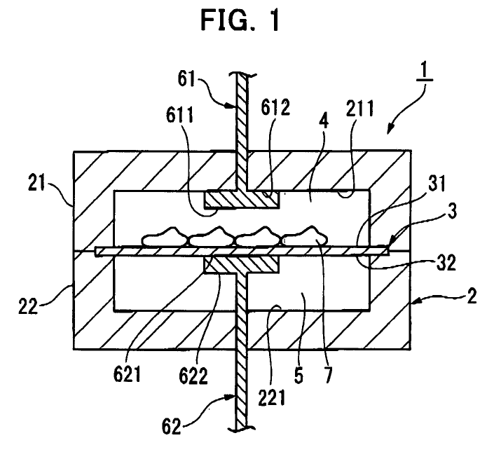

FIG. 1 is a longitudinal sectional view that shows one embodiment of an electrical signal measuring device for cells in culture (sometimes referred to below as a device) of the present invention.

A device 1 of the present invention is provided with, in a support body 2, an upper compartment 4 and a lower compartment 5 that are separated by a semipermeable membrane 3, An upper electrode 61 is provided in the upper compartment 4 via a hole that is penetrating the upper portion of the support body 2, and a lower electrode 62 is provided in the lower compartment 5 via a hole that is penetrating the lower portion of the support body 2. The upper electrode 61 and the lower electrode 62 are disposed facing each other to constitute a pair of electrodes, and so by impressing an electrical field across cells in culture 7 placed between these electrodes, electrical signals of the cells in culture can be measured.

The upper electrode 61 has a surface 611 that faces the lower electrode 62 and an opposite facing surface 612 (that is, a surface that faces the support body upper portion), is provided so as to make contact with an inner surface 211 of the support body 2 upper portion. However, in the present invention, the upper electrode 61 need not necessarily be in contact with the inner surface 211. In the range that the measurment accuracy of the electrical signals of the cells in culture 7 is not impaired, the upper electrode 61 may be provided so that the surface that faces the support body upper portion 612 of the upper electrode 61 is spaced apart from the inner surface 211.

The upper electrode 61 is preferably provided so as not to make contact with the cells in culture 7.

Also, the distance between the surface 611 of the upper electrode 61 that faces the lower electrode and a surface 31 of the semipermeable membrane 3 that faces the upper electrode 61 is preferably 1 mm or less.

By arranging the upper electrode 61 to have such a preferred disposal state, the accuracy in measuring electrical signals from the cells in culture 7 can be further enhanced.

The height of the upper compartment 4, that is, the distance between the inner surface 211 of the upper portion of the support body 2 and the surface 31 of the semipermeable membrane 3 that faces the upper electrode 61, is preferably 1 mm or less.

Also, the height of the upper compartment 4 is preferably 1.5 to 5 times, and more preferably 1.5 to 2 times, the size of the cells in culture 7.

By providing such separation, the concentration of nutritional components and oxygen that are supplied from a culture medium that refluxes through the lower compartment 5 can be maintained at a high level in the upper compartment 4. Therefore, culturing of the cells can be favorably performed by being able to make the cell culturing environment close to an actual in vivo environment.

The height of the lower compartment 5, that is, the distance between the inner surface 221 of the lower portion of the support body 2 and a surface 32 of the semipermeable membrane 3 that faces the lower electrode 62, is not particularly limited.

A facing surface 621 of the lower electrode 62 that faces the upper electrode 61 is provided to be in contact with the surface 32 of the semipermeable membrane 3 that is facing the lower electrode 62. At this time, the pressure acting on the contact surface is not particularly limited, and can be arbitrarily selected within a range in which the semipermeable membrane 3 is not damaged as long as the culture medium does not perfuse between the lower electrode 62 and the semipermeable membrane 3. Thus, by providing the lower electrode 62 to be in contact with the semipermeable membrane 3 and ensuring the culture medium does not perfuse between the lower electrode 62 and the semipermeable membrane 3, measurement of electrical signals such as impedance and the like can be performed with high sensitivity and high accuracy,

The thicknesses of the measurement surface of the upper electrode 61 and the lower electrode 62, that is, in the case of the upper electrode 61, the thickness between the surface 611 facing the lower electrode 62 and the opposite facing surface 612, and in the case of the lower electrode 62, the thickness between the surface 621 facing the upper electrode 61 and a surface 622 that faces the lower portion of the support body 2, are preferably 0.5 mm or less in either cases.

The shape of the upper electrode 61 and the lower electrode 62 are not particularly limited.

The material used for the upper electrode 61 and the lower electrode 62 is also not particularly limited provided it is able to impress an electrical field, and may be any conventional, publicly known material. Preferred materials include, for example, gold-plated brass, platinum, titanium alloy, gold, indium tin oxide (ITO) or the like.

Among these, gold-plated brass is a preferred material for the upper electrode 61 and the lower electrode 62, with the shape of the measuring surfaces of these electrodes being preferably circular with a diameter of 3 mm or less. Nail-shaped objects of such a preferred material and shape are commercially available and readily obtainable.

In the present invention, on a surface of the inner surface 211 of the support body 2 upper portion shown in FIG. 1 with which the upper electrode 61 is in contact, and/or on a surface of the semipermeable membrane 3 with which the lower electrode 62 is in contact, a metallic thin film consisting of the aforementioned material may be formed by methods such as vacuum deposition or the like, and this may be used as the electrode.

The semipermeable membrane 3 is fixed to the support body 2 so as to separate the inner compartment of the support body 2 into the upper compartment 4 and the lower compartment 5 Also, the semipermeable membrane 3 is preferably detachable from the support body 2 so as to be easily replaced when its quality becomes deteriorated.

Culturing of cells is performed on the upper compartment 4 side of the semipermeable membrane 3.

As the semipermeable membrane 3, one is used with a pore size that allows the exchange of materials such as nutritional components and oxygen in the culture medium perfusing the lower compartment 5, or waste products or the like from the cells in culture 7 in the upper compartment 4 without allowing cell in filtration into the lower compartment. Specifically, the pore diameter in the semipermeable membrane 3 is preferably 0.4 μm or greater and less than 3 μm, and preferably 0.4 μm or greater and 1 μm or less, In this way, by setting the lower limit to be 0.4 μm or greater, material exchange between the upper compartment 4 and the lower compartment 5 can be quickly performed.

Also, the material of the semipermeable membrane 3 is not particularly limited provided it is compatible with the cells in culture 7. Example materials include polyethylene, polycarbonate, polyester, and polytetrafluoroethylene or the like,

The thickness of the semipermeable membrane 3 is not particularly limited provided it is in a range that does not impair the electrical signal measurement accuracy of the cells in culture 7. But in order to perform material exchange as quickly as possible, it is preferably in a range of 10 to 20 μm.

Specifically, semipermeable membranes that are generally marketed for use in dialysis membranes and precision filtration or the like can be used.

The material of the support body 2 is not particularly limited provided it is compatible with the cells in culture 7.

A preferred material includes an oxygen permeability material that can transmit oxygen in the external air to supply to the culture medium and fluid in the device. Arbitrary oxygen permeability materials, which are conventional and publicly known, can be used as oxygen permeability materials provided they are compatible with the cells in culture 7. For example, a biocompatible oxygen permeability material that is used for oxygen permeability contact lens or the like can be used. In particular, it is more preferable if it has transparency, since the cells in culture 7 in the device are therefore observable from outside.

As an oxygen permeability material, specifically, silicone rubber with biocompatibility can be given. In particular, PDMS (polydimethylsiloxane) is preferred since it has biocompatibility and transparency, and is a low-cost material.

In the support body 2, an upper perfusion channel (not shown) and a lower perfusion channel (not shown) are provided to separately perfuse fluid in the upper compartment 4 and the lower compartment 5. These channels can be provided by connecting holes that are provided at predetermined places in the upper compartment 4 and the lower compartment 5 with piping or the like (not shown). The material of the piping is not particularly limited.

In order to perfuse the culture medium required for a cell culture as a fluid in the lower compartment 5 to supply the nutritional components, oxygen or the like to the cells in culture 7 via the semipermeable membrane 3, and eject the discharged waste from the cells in culture 7 outside of the lower compartment 5, in the present invention it is preferable to separately provide a culture medium feeding channel and a culture medium discharging channel in the lower compartment 5 as the lower perfusion channel.

Also, the composition of the culture medium may be arbitrarily selected in accordance with the type of cells to be cultured.

Also, in the upper compartment 4, the fluid can be perfused continuously or intermittently via the upper perfusion channel. By perfusing the fluid in the upper compartment 4, flowage is imparted to the cells in culture 7 on the semipermeable membrane 3. Thereby, the environment of the cell culture region can approximate an actual in vivo environment, and so culturing of cells can be favorably performed. Also, monitoring of the culturing environment (pH, glucose concentration, physiological active substance concentration or the like) of the cells in culture 7 can be performed via the upper perfusion channel.

The fluid that is perfused in the upper component 4 is not particularly limited provided it does not have an adverse effect on the cells in culture 7, however a culture medium is preferred.

The types of cells in culture 7 whose electrical characteristics are to be measured with the device 1 of the present invention are not particularly limited, and can be arbitrarily selected in accordance with the objective. For example, intestinal Caco-2 cells are suitable for the present invention.

The device of the present invention can be used to measure the electrical characteristics of cells in culture by electrically connecting the upper electrode 61 and the lower electrode 62 to external electrodes (not shown). For example, by connecting these electrodes to a direct-current power supply, the electrical resistance of the cells in culture can be measured, and by connecting to an alternating-current power supply, the impedance of the cells in culture can be measured. That is, it is possible to select the external electrodes to be connected depending on the purposes.

Since the device 1 of the present invention is extremely small and the structure is also simple, for example, it can be used as an electrical signal measurement array by providing a plurality thereof on the same base material. The type of base material, the method of forming the array, and the mode of the array are not particularly limited and can be selected depending on the purposes. This type of electrical signal measurement array is suitably used for screening numerous samples or the like.

The device 1 of the present invention can be manufactured as described below. That is, an upper support body 21 of a predetermined shape, in which a hole for insertion of the upper electrode 61 and a hole for connection of the upper perfusion channel are provided, and a lower support body 22 of a predetermined shape, in which a hole for insertion of the lower electrode 62 and a hole for connection of the lower perfusion channel are provided, are manufactured. Next, after inserting the upper electrode 61 in the upper support body 21, and inserting the lower electrode 62 in the lower support body 22, the edges of the upper support body 21 and the lower support body 22 are glued together sandwiching the semipermeable membrane 3 therebetween so that the upper electrode 61 and the lower electrode 62 are facing each other. At this time, the lower electrode 62 is positioned so that the surface of the lower electrode facing the upper electrode 621 is in contact with the semipermeable membrane 3 without gaps therebetween. Then, piping is connected to the hole for connection to the upper perfusion channel and to the hole for connection to the lower perfusion channel, and further, the upper perfusion channel and the lower perfusion channel are provided in the device body 2, the device 1 of the present invention is obtained.

The device 1 of the present invention has a simple structure as described above, and since commercially available nail-shaped objects can be employed as the upper electrode 61 and the lower electrode 62, the device 1 can be readily manufactured.

By using the device 1 of the present invention, electrical characteristics of cells in culture can be measured as follows. Namely, cells are introduced to a location between the upper electrode 61 and the lower electrode 62 which is on the upper compartment 4 side of the semipermeable membrane 3, and while separately perfusing a fluid in the upper compartment 4 and a culture medium in the lower comment 5, the cells on the semipermeable membrane 3 are cultured. By impressing an electrical field on the cells in culture 7 from the upper electrode 61 and the lower electrode 62, the current response of the cells in culture 7 obtained as a response is measured. If an electrical field is impressed by connecting the device 1 of the present invention to an external direct-current power supply, the electrical resistance of the cells in culture can be measured, and if connected to an alternating-current power supply, the impedance of the cells in culture can be measured. By measuring the impedance, more information can be obtained relating to the cells in culture.

The conditions for impressing an electrical field on the cells in culture are not particularly limited, and may be arbitrarily selected in accordance with the objectives of the measurement and the type of the cells in culture.

When performing electrical signal measurement, the cells in culture can be measured in real time without the need to stop the culturing of the cells. For example, the growing process of cells in culture and the process of tissue formation such as membranous structure or the like of cells in culture can be measured in real time. Also, since the perfusion of fluid in the upper compartment 4 and the perfusion of the culture medium in the lower compartment 5 are separately performed, if a drug or the like is added to the upper compartment 4, the response of the cells in culture to this drug or the like can also be measured in real time.

In the device 1 of the present invention, by perfusing the fluid in the upper compartment 4 and perfusing the culture medium in the lower compartment 5 as described above, simultaneously with nutritional components and oxygen being supplied from the lower compartment 5 to the cells in culture 7 via the semipermiable membrane 3, waste products or the like discharged from the cells in culture 7 are recovered to the lower compartment 5 via the semipermiable membrane 3. That is, nutritional components and oxygen are supplied from a given direction and waste products or the like discharged from the cells in culture 7 are recovered from a certain direction. Thereby, it is possible to achieve an environment resembling an actual in vivo environment in which the supply of nutritional components and oxygen and the discharge of waste products or the like are performed via blood vessels or the like. Accordingly, culturing of cells can be favorably performed.

Here, perfusion as used here refers simply to the flowing of a fluid which includes so-called reflux in which a fluid flows in a given direction, and a flow in which a fluid flows with sequentially changing the flowing direction. However, it is preferable to continuously reflux the culture medium in a certain direction by separately providing a culture medium feeding channel and a culture medium discharging channel as the afore-described lower perfusion channel in the lower compartment 5.

Doing so can efficiently supply nutritional components and oxygen to the cells in culture 7 and discharge the waste products or the like eliminated from the cells in culture 7 to the outside, and therefore culturing of the cells is more favorably performed.

The flow rate of the fluid in the upper compartment 4 and the flow rate of the culture medium in the lower compartment 5 are not particularly limited provided they are in a range that does not inhibit culturing of the cells and does not impair the accuracy of measuring electrical signals of the cells in culture.

The culture medium that is perfused in the lower compartment 5 is preferably replaced at least every 3 or 4 days in order to remove waste products and secretions or the like discharged from the cells in culture.

Other cell culturing conditions and culturing methods may be arbitrarily selected in accordance with the type of cells being used, and so are not particularly limited.

As described above, in the device of the present invention, by providing the lower electrode to be in contact with the semipermeable membrane and ensuring that the culture medium does not perfuse between the lower electrode and the semipermeable membrane, electrical signals such as impedance and the like can be measured with high sensitivity and high accuracy.

Since the perfusion of fluid in the upper compartment and the perfusion of the culture medium in the lower compartment are separately performed, if a drug or the like is added to the upper compartment, the response of the cells in culture to this drug or the like can also be measured in real time.

By making the height of the upper compartment low, preferably 1 mm or less, cells on the semipermeable membrane can be favorably cultured.

Since the device of the present invention is extremely small and the structure is also simple, it can be readily used as an electrical signal measurement array by providing a plurality thereof on the same base material, and is therefore suitably used for screening or the like numerous samples.

By using the device of the present invention, measurement of not only resistance values but also impedance is possible, and cells in culture on a semipermeable membrane can be analyzed noninvasively and in real time, so that greater amount of information is obtained.

### EXAMPLES

Hereinbelow, the present invention is described in greater detail with specific examples. Note that the present invention is in no way limited to these examples shown below.

### Embodiment 1

### Confirmation of the Formation of a Membranous Structure in the Cells in Culture by Impedance Measurement

While culturing intestinal Caco-2 cells using the device of the present invention shown in FIG. 1, impedance of the cells was measured.

The device used is specifically described as follows. That is, the support body is made of PDMS, and the size of the compartment in the support body (the combined size of the upper compartment and the lower compartment) is 15 mm in length, 6 mm in width, and 1.6 mm in height. This compartment is divided into an upper compartment and a lower compartment each with a height of approximately 0.8 mm using a polyester porous membrane which is provided with a diameter of 0.4 μm and a thickness of 14 μm (code 3450, made by CORNING INC.).

Gold-plated brass nail-shaped objects with a length of 15 mm, an external diameter of 0.5 mm, and its circular head portion with a thickness of 0.4 mm and a diameter of 1.2 mm are used as the upper electrode and the lower electrode using a flat surface of the head portion as a measuring surface. Also, the upper electrode is fixed to the support body with its surface opposite to the surface facing the lower electrode in contact with the inside surface of the upper support body, while the lower electrode is fixed to the support body with its surface facing the upper electrode in contact with the semipermeable membrane. At this time, the distance between the surfaces of the upper electrode that faces the lower electrode and the lower electrode is 0.4 mm. Then, these electrodes are connected to an alternating-current power supply.

The culturing conditions of the cells and impedance measurement conditions are as follows.

Culturing conditions: As the culture medium, 10% fetal bovine serum, 1% MEM nonessential amino acid solution, 20 mM HEPES buffer solution, 100N/mL penicillin, 100 μg streptomycin, and 1 μg amphotericin added to DMEM (Dulbecco's Minimum Essential Medium) is used, and culturing is performed at an ambient temperature of 37 degrees C., with the carbon dioxide concentration maintained at 5%.

Measurement condition: Measurement is performed by using a commercially available impedance analyzer, by applying an AC electrical field of 10 mV amplitude (1 Hz to 10 MHz) across the electrodes.

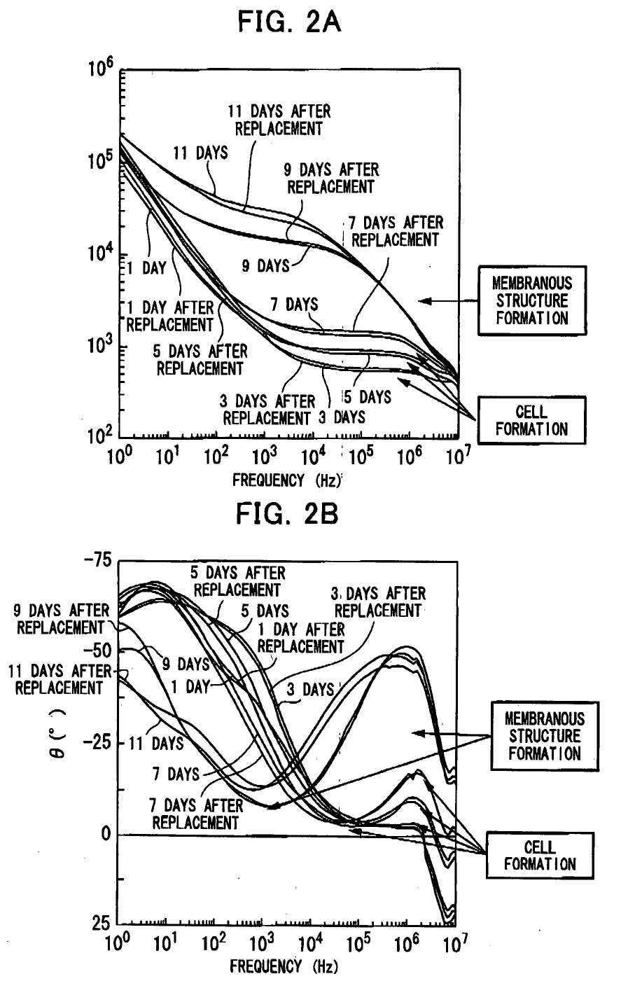

The culture medium was replaced every two days, with the impedance measurement being performed before and after the culture medium replacement. In the legend shown in FIG. 2, “3 days” means prior to the culture medium replacement on the third day from the start of culturing, while “3 days after replacement” means after he culture medium replacement on the third day from the start of culturing.

FIG. 2A shows the amplitude spectra of the measured impedance, with the vertical axis of the graph representing absolute values of the amplitude of the impedance. From FIG. 2A, cell growth is confirmed until the seventh day from the start of culturing From the ninth day onward, a notable increase (18KΩ) in the impedance amplitude was observed, and a formation of a tight junction between cells and a formation of a membranous structure by the cells were confirmed.

Also, FIG. 2B shows the phase spectra of the measured impedance, with the vertical axis of the graph representing the phase (θ). From FIG. 2B, it is observed that, until the seventh day from the start of culturing, the curve shifts to the lower frequency side and a characteristic peak around 10⁶ Hz region appears while cell is confirmed to be in growth during this period. From the ninth day onward, a notable shift of the curve to the lower frequency side and a notable decrease in the phase (θ=−50 degrees) around the 10⁶ Hz region were observed, and the formation of a membranous structure in the cells in culture was confirmed.

From the above, the impedance of cells in culture could be measured with high accuracy and in real time by using the device of the present invention, and cellular growth, the formation of a tight junction between cells, and the formation of a membranous structure could be accurately confirmed.

### Example 2

### Confirmation of the Effect of Adding Cupric Chloride (CuCl₂) to Cells in Culture by Impedance Measurement

Using the device used in Example 1, while culturing intestinal Caco-2 cells to which cupric chloride is added; impedance of the cells was measured.

The culturing conditions of the cells and impedance measurement conditions are the same as in Example 1.

The culture medium was replaced every two days, with the impedance measurement being performed before and after the culture medium replacement.

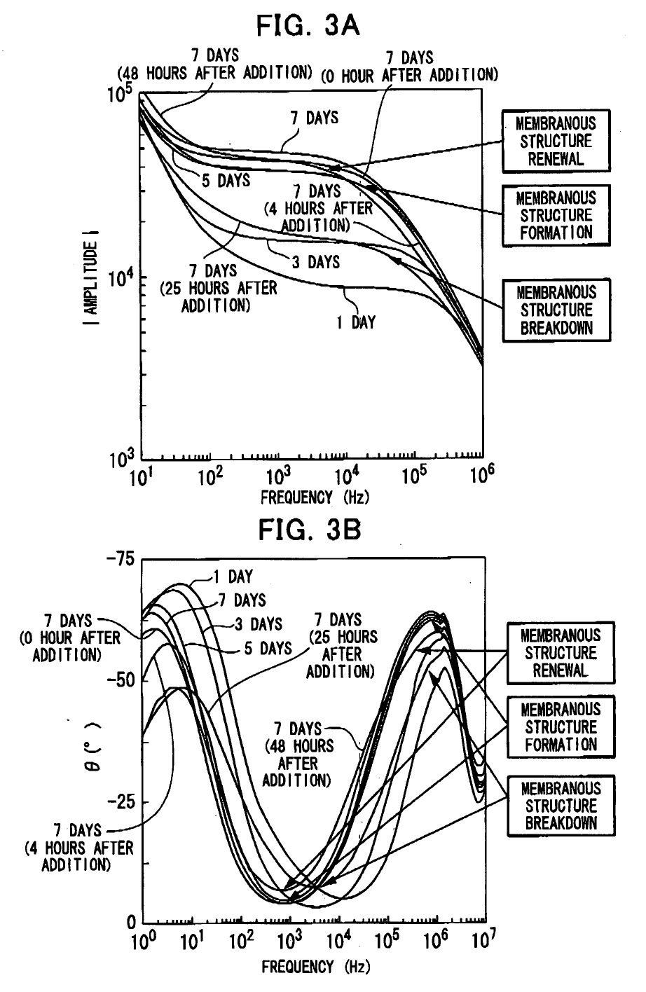

Culturing was normally performed until the seventh day from the start of culturing. After impedance measurement, cupric chloride was added to a concentration of 30 μM to the culture medium, and the cell culturing was continued as it was from the moment directly after the cupric chloride was added (0 hours) to four hours after the addition. After the passage of four hours, the impedance was measured, and the culture medium containing the cupric chloride was washed away by a culture medium not containing the cupric chloride, accordingly washing was performed while the cells in culture were remained in the device. During this period, the impedance of the cells in culture was arbitrarily measured. The result is shown in FIG. 3. In the legend shown in FIG. 3, “3 days” means the third day from the start of culturing, while “7 days (4 hours after addition)” means four hours after addition of cupric chloride to the culture medium on the seventh day from the start of culturing.

FIG. 3A shows the amplitude spectra of the measured impedance, with the vertical axis showing absolute values of the amplitude of the impedance. On the fifth day after the start of culturing, the impedance amplitude was observed to increase notably, reaching maximum on the seventh day. That is, a formation of a membranous structure in the cells in culture was confirmed between the third and fifth day after the start of culturing.

On the seventh day, until four hours from the addition of cupric chloride, hardly any changes in the impedance amplitude were observed, and so the membranous structure was confirmed to be functioning. After washing the cells in culture and then returning the cells in culture to the device, the effect of adding cupric chloride appeared, and at 25 hours after the addition, a reduction in the impedance amplitude was observed and so the breakdown of the membranous structure was confirmed. At 48 hours after the addition, an increase in the impedance amplitude was again observed, and so renewal of the membranous structure was confirmed. In other words, it was confirmed that the effect of adding cupric chloride on the membranous structure of the cells in culture disappears with the passage of time.

Also, FIG. 3B shows the phase spectra of the measured impedance, with the vertical axis of the graph representing the phase (θ). From the third day to the seventh day after the start of culturing, a shift in the curve to the lower frequency side and a characteristic decrease in the phase θ (θ=−62 degrees) around 10⁶ Hz region was observed, and so a formation of a membranous structure in the cells in culture was confirmed.

On the seventh day, until four hours from the addition of cupric chloride, the membranous structure was confirmed to be functioning as it was without large changes in the curve. Then, after washing the cells in culture and then returning the cells in culture to the device, the effect of adding cupric chloride appeared, and at 25 hours after the addition, a shift in the curve to the higher frequency side and an characteristic increase in the phase θ (θ=−53 degrees) around 10⁶ Hz region were observed and so the breakdown of the membranous structure was confirmed. Moreover, 48 hours after the addition, a new shift in the curve to the lower frequency side and a characteristic decrease in the phase θ (θ=−58 degrees) around 10⁶ Hz region were observed, and so renewal of the membranous structure was confirmed. That is, an effect of adding cupric chloride on the membranous structure of the cells in culture was confirmed to disappear with the passage of time by measuring the impedance of the cells in culture with high accuracy and in real time using the device of the present invention

The present invention is suited for use in an extremely wide range of fields, such as pharmacokinetic analysis and screening of candidate substances for medicines, which play important roles in the drug development process of medicines, and measurement of environmental toxicity of chemicals, in addition to optimization of the culturing conditions of cells and observation of the state of cells in culture.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An electrical signal measuring device for cells in culture comprising:
a support body;
a compartment in the support body;
a semipermeable membrane; and
electrodes, wherein
the compartment is divided by the semipermeable membrane into an upper compartment and a lower compartment;
an upper electrode is provided in the upper compartment, and a lower electrode that faces the upper electrode and whose facing surface is in contact with the semipermeable membrane is provided in the lower compartment; and
an upper perfusion channel and a lower perfusion channel are provided in the support body to separately perfuse fluid in the upper compartment and the lower compartment.

2. The electrical signal measuring device for cells in culture according to claim 1,
wherein the distance between the surface of the upper electrode that faces the lower electrode and the surface of the semipermeable membrane that faces the upper electrode is 1 mm or less.

3. The electrical signal measuring device for cells in culture according to claim 1 or claim 2,
wherein the thickness of the measurement surface of the upper electrode and the lower electrode is 0.5 mm or less.

4. The electrical signal measuring device for cells in culture according to claim 1,
wherein the material of the upper electrode and the lower electrode is gold-plated brass, and the shape of the measuring surface of these electrodes are circular with a diameter of 3 mm or less.

5. The electrical signal measuring device for cells in culture according to claim 1,
wherein the material of the support body is polydimethylsiloxane.

6. The electrical signal measuring device for cells in culture according to claim 1 that is an impedance measuring device.

7. An electrical signal measuring array for cells in culture,
wherein the electrical signal measuring device for cells in culture according to claim 1 are plurality provided on a base material.

8. A method of measuring electrical characteristics of cells in culture using the electrical signal measuring device for cells in culture according to claim 1, comprising:
introducing cells into a location located between the upper electrode and the lower electrode on the upper compartment side of a semipermeable membrane;
culturing the cells on the semipermeable membrane while separately perfusing a fluid in the upper compartment and a culture medium in the lower compartment;
impressing an electrical field on the cells in culture by the upper electrode and the lower electrode; and
measuring the current response of the cells in culture to the impressed electrical field.
